(19) United States
(12) Patent Application Publication
(10) Pub. No.: US 2025/0332321 A1
(43) Pub. Date: Oct. 30, 2025

(54) ELECTROACTIVE TITANIUM SUPPORT-REINFORCED COMPOSITE FILM AND METHOD FOR PREPARING SAME

(71) Applicants: PEKING UNIVERSITY SCHOOL OF STOMATOLOGY, Beijing (CN); BEIJING PIEZO-DENT MEDICAL TECHNOLOGY CO., LTD, Beijing (CN)

(72) Inventors: Xuehui ZHANG, Beijing City (CN); Xuliang DENG, Beijing City (CN); Yunyang BAI, Beijing City (CN); Shenpo YUAN, Beijing City (CN)

(21) Appl. No.: 18/871,475

(22) PCT Filed: Sep. 7, 2022
(86) PCT No.: PCT/CN2022/117448

§ 371 Date: Dec. 3, 2024

(30) Foreign Application Priority Data
Jun. 9, 2022 (CN) 202210643934.1

(51) Int. Cl. A61L 27/06; A61F 2/28; A61F 2/30; A61L 27/50

(52) U.S. Cl. CPC A61L 27/06; A61F 2/2803; A61F 2/2846; A61F 2/2875; A61F 2/3094; A61L 27/50; A61F 2002/2821; A61F 2310/00023

(57) ABSTRACT

An electroactive titanium support-reinforced composite film includes a titanium support and a film material coating the titanium support. The titanium support has a structure designed according to securing sites. The electroactive titanium support-reinforced composite film of the present invention can be bent and shaped for a close fit with a hard tissue, has excellent mechanical performance and a stable electroactive bending strength of bionic magnitude, and can prevent the collapse of surrounding tissues and tissue adhesion, effectively maintain a three-dimensional space for bone osteanagenesis and effectively promote bone injury healing, featuring ease and convenience of clinical operation and capability of promoting bone marrow mesenchymal stem cell adhesion, cytoskeleton rearrangement and induced osteogenic differentiation. The support-reinforced composite film is suitable for mandible or cranium injury repair in different ranges and has significant efficacy for clinical indications such as alveolar bone vertical bone augmentation and alveolar ridge preservation after tooth extraction.

The present application is a U.S. National Phase of International Application Number PCT/CN2022/117448 filed on Sep. 7, 2022, which claims the priority to the Chinese patent application No. CN202210643934.1 filed on Jun. 9, 2022 with the Chinese Patent Office and entitled “ELECTROACTIVE TITANIUM SUPPORT-REINFORCED COMPOSITE FILM AND METHOD FOR PREPARING SAME”, the disclosures of which are incorporated herein by reference in their entirety.

### TECHNICAL FIELD

The present disclosure relates to the technical field of orthopedic and oral surgery implantable repair materials, and particularly relates to an electroactive titanium scaffold-reinforced composite film used for mandible defect repair, alveolar bone augmentation, or cranial repair, and a preparation method therefor.

### BACKGROUND

Guided bone regeneration (GBR) is the most widely used bone augmentation technique in oral surgery and orthopedic surgery. The basic principle is that a barrier film is used to effectively prevent epithelial cells or fibrocytes from entering a bone defect area, maintain a defect space, and promote bone defect repair. However, materials conventionally used as barrier films (e.g., absorbable collagen films or non-absorbable PTFE films) lack mechanical strength, are difficult to maintain a stable space, and may fold and collapse after surgery, affecting bone regeneration. In cranioplasty, the choice of repair materials is crucial. At present, repair materials commonly used in clinics are mainly classified into autologous bones, allogeneic bones, hydroxyapatite materials, metallic titanium materials, polymer materials, and the like. The autologous bone repair is limited in clinical use due to the need to open up a second surgical area, the limited source, difficulty in shaping, and easy absorption. The allogeneic bones and xenogeneic bones are also abandoned due to significant rejection and high infection rates. The hydroxyapatite materials have good biocompatibility and osteoinductivity, but have poor mechanical strength and low tensile strength, which makes it prone to breakage by screw fixation during surgery and external forces after surgery, resulting in high infection rates after surgery.

As for the metallic titanium materials, although they have good biocompatibility and mechanical strength, due to cutting injury, poor heat insulation, and difficulty in shaping, they often cause complications such as rejection, infection, pain, and collapse and deformation after surgery, and interfere with nuclear magnetic resonance examination. Therefore, polymer cranial repair materials have emerged. Polymethyl methacrylate is brittle and fragile, and has insufficient bioactivity, and high-density polyethylene has low toughness and hardness, and insufficient support capability, both requiring further development.

At present, the polymer material commonly used in clinics is mainly polyetheretherketone (PEEK), which has good biocompatibility, X-ray transmission performance, and biomechanical properties similar to those of a cortical bone. However, PEEK is too expensive, lacks osseointegration, cannot be combined with surrounding autologous cranial bone, and has a high rejection risk.

A traditional titanium mesh is used to repair large-area bone defects in clinics at home and abroad. However, in the case of bone implant augmentation surgery and extensive bone defects, exposure is prone to occur after surgery, leading to infection and failure of the surgery. Therefore, the development of a reinforced composite film with an electroactive titanium scaffold is an important requirement of the current guided bone regeneration technology.

The information in the background is only for the purpose of illustrating the general background of the present disclosure and should not be taken as an acknowledgment or any form of suggestion that such information forms the prior art that is already known to those of ordinary skill in the art.

### SUMMARY

In order to solve the technical problems in the prior art, the present disclosure provides an electroactive titanium scaffold-reinforced composite film and a preparation method therefor. The electroactive titanium scaffold-reinforced composite film provided by the present disclosure has good performance in both macroscopic properties and microscopic structure, and provides a sufficient three-dimensional space for new bone regeneration in a bone repair process, promoting osteogenesis. In addition, the electroactive titanium scaffold-reinforced composite film can be bent and shaped according to different tooth positions for a tight fit with a corresponding alveolar bone hard tissue. The film simultaneously exhibits excellent mechanical properties and stable biomimetic electroactivity, which can promote bone marrow mesenchymal stem cell adhesion, cytoskeleton rearrangement, and osteogenic differentiation, thereby significantly improving effects of vertical bone augmentation. Specifically, the present disclosure comprises the following.

In a first aspect of the present disclosure, provided is an electroactive titanium scaffold-reinforced composite film, wherein the composite film has a quadrilateral or substantially quadrilateral profile, and a fixing site for fixing the composite film is arranged at each corner of the quadrilateral or a vicinity thereof;
the composite film comprises: a titanium scaffold and a film material coating the titanium scaffold, wherein the titanium scaffold consists of a titanium-based material with a thickness of 20-500 μm and has a structure designed according to the fixing sites;
the titanium scaffold comprises: a main frame and secondary frames attached to both sides of the main frame with a bifurcation structure, wherein the secondary frame comprises a first branched structure and a second branched structure at a certain angle, and ends of the bifurcation structure are located at the fixing sites or located at positions near the fixing sites.

According to the electroactive titanium scaffold-reinforced composite film of the present disclosure, preferably, a polymer material layer comprises a first layer and a second layer, and the titanium scaffold is coated with the first layer and the second layer, and an area ratio (coverage area ratio) of the titanium scaffold in the composite film is 0.6-1.

According to the electroactive titanium scaffold-reinforced composite film of the present disclosure, preferably, the main frame extends along a length direction, the secondary frame extends along a width direction, the main frame is an elongated strip-shaped structure, and the angle is 20-30 degrees, thereby forming the titanium scaffold into a dumbbell shape with a thin middle part and two wide ends.

According to the electroactive titanium scaffold-reinforced composite film of the present disclosure, preferably, the titanium scaffold has a symmetrical structure along the length direction and the width direction.

According to the electroactive titanium scaffold-reinforced composite film of the present disclosure, preferably, an aspect ratio of the titanium scaffold is 2-4:1, and a ratio of the length of the main frame to the width of the secondary frame is 0.9-2:1.

According to the electroactive titanium scaffold-reinforced composite film of the present disclosure, preferably, the titanium scaffold further comprises a transverse frame located in the middle of the main frame and substantially perpendicular to the main frame.

According to the electroactive titanium scaffold-reinforced composite film of the present disclosure, preferably, the secondary frame further comprises a third branched structure located between the first branched structure and the second branched structure, and the third branched structure extends along a direction of the main frame, thereby forming the titanium scaffold into a pozidriv shape.

According to the electroactive titanium scaffold-reinforced composite film of the present disclosure, preferably, the titanium scaffold further comprises two transverse frames located at both ends of the main frame, respectively, and substantially perpendicular to the main frame, thereby forming the titanium scaffold into a glider shape.

According to the electroactive titanium scaffold-reinforced composite film of the present disclosure, preferably, the main frame and the branched structures of the secondary frame have the same width.

Preferably, the composite film is obtained by compositing the titanium scaffold inside the polymer material layer, annealing, and corona polarizing. More preferably, the first layer and the second layer each consists of identical or different compositions and are each independently selected from at least one of polyester, polyvinylidene fluoride PVDF, poly(vinylidene fluoride-trifluoroethylene) P(VDF-TrFE), polymethyl methacrylate PMMA, and polydimethylsiloxane.

Preferably, the composite film has a thickness of 100-500 μm, preferably 100-400 μm, more preferably 100-300 μm, such as 250 μm.

In a second aspect of the present disclosure, provided is a preparation method for the electroactive titanium scaffold-reinforced composite film according to the first aspect, which comprises the following steps:
(1) compositing a titanium scaffold inside a polymer material layer to form a film structure, and arranging fixing sites at positions corresponding to ends of bifurcation structures of the titanium scaffold;
(2) increasing a temperature to 105-145° C., preferably 110-130° C., and more preferably 120-130° C., at a rate of 2.5-4° C./min, keeping the temperature for 30-80 min, preferably 40-70 min, and more preferably 60 min, and then performing cooling, preferably natural cooling, to room temperature; and
(3) performing polarization treatment using a polarization method, with polarization treatment parameters comprising a polarization field intensity of 0.1-10 kV/mm and a polarization time of 10-60 min, such that the electroactive titanium scaffold-reinforced composite film can be obtained.

Beneficial effects of the present disclosure include, but are not limited to, the following:
(1) According to the present disclosure, by optimizing the structure of the titanium scaffold, under the condition of ensuring the mechanical strength, the thickness and the area of the titanium scaffold are reduced, the exposure risk on a mucosa is reduced, and the uniformity of the support strength and the plasticity is realized. Moreover, the optimized titanium scaffold is an elongated fork-shaped structure, which significantly enhances the mechanical properties of the composite film, including the tensile strength and the elastic modulus, and reduces the bending strength, which is beneficial for improving the service performance and long-term stability of the material.
(2) The composite film of the present disclosure can be bent and shaped according to the shapes of the alveolar bones corresponding to different tooth positions for a tight fit with a corresponding alveolar bone hard tissue, and the edge line of the titanium scaffold is much shorter than that of the titanium mesh, such that the exposure risk of the titanium scaffold is greatly reduced after bending.
(3) The composite film of the present disclosure, through annealing and high-voltage electric field polarizing, has a biomimetic electric potential on the surface of the composite film and good stability of charge, constructs a biomimetic electrical microenvironment in the bone defect area, and promotes bone repair or vertical bone augmentation.
(4) The composite film of the present disclosure exhibits excellent anti-tissue adhesion properties. Particularly, animal experimental results show that after the composite film is conveniently removed from the specimens for CT and histological examinations, the integrity of the repaired tissue is still maintained, and there is no residual tissue on the surface of the composite film. This indicates that the composite film can effectively prevent tissue adhesion, thereby overcoming the shortcomings in the prior art that pure titanium meshes or existing expanded polymer repair film materials are prone to adhere to tissues.

### BRIEF DESCRIPTION OF THE DRAWINGS

FIG. 1 is a structural schematic diagram of an exemplary dumbbell-shaped titanium scaffold of the present disclosure.

FIG. 2 is a structural schematic diagram of another exemplary pozidriv-shaped titanium scaffold of the present disclosure.

FIG. 3 is a structural schematic diagram of another exemplary glider-shaped titanium scaffold of the present disclosure.

FIGS. 4-6 show three-dimensional finite element analysis results of titanium scaffolds of different shapes.

FIG. 7 shows the mechanical property characterization results of titanium scaffolds of different shapes.

FIGS. 8-11 show simulation results of area ratio optimization of titanium scaffolds in polymer matrices.

FIG. 12 is a physical diagram of the titanium scaffolds of the present disclosure.

FIG. 13 is a physical diagram of the electroactive titanium scaffold-reinforced composite film of the present disclosure.

FIG. 14 shows the test results of piezoelectric constants of the electroactive titanium-reinforced composite films with different thicknesses and different annealing times.

FIG. 15 shows the comparison results of the piezoelectric constants of the titanium-reinforced composite films with different interface treatments.

FIGS. 16-17 show the mechanical property characterizations of the electroactive titanium-reinforced composite film materials (FIG. 16: tensile strength; FIG. 17 left: elastic modulus; right: bending strength).

FIGS. 18-20 show the evaluation results of the electrical responsiveness of the electroactive titanium-reinforced composite film materials (FIG. 18 polarized; FIG. 19 unpolarized; FIG. 20 polarized versus unpolarized).

FIG. 21 shows the time-series monitoring results of the piezoelectric constant of the titanium-reinforced composite film.

FIG. 22 shows immunofluorescence images of focal adhesions, wherein white dotted lines indicate the nucleus, a column of images of focal adhesions are shown as focal adhesion fluorescence images, and a column of images of F-actins are shown as cytoskeleton fluorescence images.

FIG. 23 shows the quantitative analysis results of the cell area and the area and the number of focal adhesions (*p<0.05, **p<0.01, ***p<0.001).

FIG. 24 shows the immunofluorescence results of BMP-2 expression by BMSCs osteogenic differentiation induced by the electroactive titanium-reinforced composite films.

FIG. 25 shows the results of osteogenic gene expression in bone marrow mesenchymal stem cells promoted by the electroactive titanium-reinforced composite film (*p<0.05, **p<0.01, ***p<0.001).

FIG. 26 shows a surgical procedure for alveolar bone augmentation in beagles.

FIG. 27 shows the μCT results 1 month after implantation of the electroactive titanium scaffold-reinforced composite film.

FIG. 28 shows the μCT results 3 months after implantation of the electroactive titanium scaffold-reinforced composite film.

FIG. 29 shows the μCT quantitative analysis results of vertical bone augmentation promoted by the electroactive titanium scaffold-reinforced composite film (* and # indicate statistically significant differences compared with the blank group and the Ti-P(VDF-TrFE) group, respectively, p<0.05).

FIG. 30 shows H&E staining results 3 months after implantation of the titanium-reinforced composite film, wherein a, b, c, and d are high magnification fields of view of circled areas. (a) The left side of an alveolar ridge crest; (b) the top of an alveolar ridge crest; (c) the right side of an alveolar ridge crest; and (d) the center of an alveolar ridge. (nb: new bone; ob: old bone. Magnification×100).

FIG. 31 shows Masson staining results 3 months after implantation of the titanium-reinforced composite film, wherein a, b, c, and d are high magnification fields of view of circled areas. (a) The left side of an alveolar ridge crest; (b) the top of an alveolar ridge crest; (c) the right side of an alveolar ridge crest; and (d) the center of an alveolar ridge. (nb: new bone; ob: old bone. Magnification×100).

FIG. 32 is a structural diagram of a commercially available titanium mesh composite film.

FIGS. 33-36 show the comparison results of mechanical properties between the commercially available titanium mesh composite film and the scaffold composite films of the present disclosure. The data show that the tensile modulus, elastic limit, and elastic modulus of the titanium scaffold composite films are higher than those of the commercial titanium mesh composite film. As a result, during clinical surgical operations and bone healing processes, the titanium scaffold composite film is less likely to experience delamination between the metal and the organic polymer, ensuring the overall integrity of the composite material and the stability of clinical surgical operations, enabling a predictable bone augmentation process. The lower bending modulus indicates that it is easier to shape the material according to clinical requirements and bone morphology.

### BRIEF DESCRIPTION OF THE REFERENCE NUMERALS

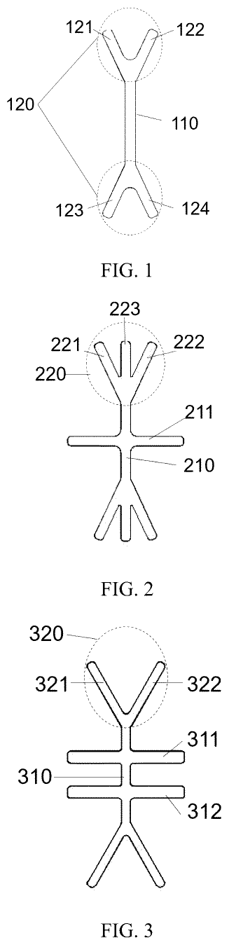

dumbbell—shaped titanium scaffold of FIG. 1:110—main frame, 120—secondary frame, 121—first branched structure, 122—second branched structure, 123—third branched structure, and 124—fourth branched structure;
pozidriv—shaped titanium scaffold of FIG. 2:210—main frame, 211—transverse frame, 220—secondary frame, 221—first branched structure, 222—second branched structure, and 223—third branched structure; and
glider—shaped titanium scaffold of FIG. 3:310—main frame, 311—first transverse frame, 312—second transverse frame, 320—secondary frame, 321—first branched structure, and 322—second branched structure.

### DETAILED DESCRIPTION

Various exemplary embodiments of the present disclosure are described in detail below, which should not be construed as limitations to the present disclosure but as a more detailed description of certain aspects, features, and embodiments of the present disclosure.

It should be understood that the terms used herein are for the purpose of illustrating particular embodiments only, rather than limiting the present disclosure. In addition, for numerical ranges in the present disclosure, it should be understood that the upper and lower limits of the range and each intervening value therebetween are specifically disclosed. Every smaller range between any stated value or intervening value in a stated range and any other stated or intervening value in that stated range is encompassed within the present disclosure. The upper and lower limits of such smaller ranges may independently be included or excluded in the range.

Unless defined otherwise, all technical and scientific terms used herein have the same meaning as commonly understood by those of ordinary skill in the art. Although only preferred methods and materials are described herein, any methods and materials similar or equivalent to those described herein can be used in the practice or testing of the present disclosure. All documents described herein are incorporated by reference to disclose and describe the methods and/or materials associated with the documents. In case of conflict with any incorporated document, the specification shall prevail. Unless defined otherwise, “%” is a percentage based on weight.

Herein, the term “titanium scaffold” refers to a scaffold structure that is located within a composite film, in use, for supporting the composite film. It is known that mechanical properties in the composite film are affected by the titanium scaffold. Generally, the smaller the area ratio of the titanium scaffold in the composite film, the poorer the mechanical support properties of the composite film. The titanium scaffold of the present disclosure has the minimum area ratio through optimization, and simultaneously has the optimal mechanical support. The titanium scaffold consists of the titanium-based material in the form of a titanium sheet, and as long as a desired elastic modulus and a desired bending strength can be achieved under the condition of an ultra-thin thickness, there is no particular limitation on the titanium-based material. However, a pure titanium sheet or a titanium alloy is preferred. The titanium in the pure titanium sheet generally has a purity of no less than 99.90%, preferably no less than 99.95%, and more preferably no less than 99.99%. Examples of such pure titanium sheets include, but are not limited to, grade four pure titanium plates and grade five pure titanium plates. Examples of the titanium alloys include, but are not limited to, titanium zirconium alloys, titanium magnesium alloys, and the like.

Herein, the titanium scaffold has a thinner thickness compared with the commonly used titanium sheet in current guided bone regeneration. Generally, the thickness is 10-300 μm, for example, 20-200 μm, 20-250 μm, preferably 25-150 μm, such as 100 μm, 80 μm, and 50 μm. At present, a pure titanium mesh for medical use generally has a thickness of no less than 200 μm, while the titanium scaffold of the present application can have a thickness of no more than 100 μm, preferably no more than 80 μm or no more than 50 μm, more preferably no more than 30 μm, and further preferably no more than 20 μm. In another aspect, the thickness generally needs to be more than 10 μm to provide the desired mechanical properties and to ensure that the deformation stress is substantially consistent with that of the polymer material, thereby achieving a high degree of fit with the polymer material layer. If the titanium scaffold of the present disclosure has an excessive thickness, in one aspect, the titanium scaffold is not easy to suture, and the possibility of exposure from soft tissues is increased, leading to infection. In another aspect, the bending strength is increased, and after the titanium scaffold is composited with the polymer film, the deformation stress of the titanium scaffold is inconsistent with that of the polymer film, such that the polymer film cannot effectively wrap the scaffold, making the titanium scaffold prone to delamination with the polymer film in use.

Herein, the term “desired elastic modulus” refers to an elastic modulus enabling effective bending during the mandible defect repair. Meanwhile, the elastic modulus range is equivalent to the modulus of the polymer material used in defect repair. The modulus is generally 0.05-0.5 GPa, preferably 0.1-0.4 GPa, and more preferably 0.2-0.35 GPa. Here, the elastic modulus is measured using a universal tester. If the elastic modulus is too low, it is not conducive to maintaining the defect space during the mandible defect repair, which is not conducive to the bone defect repair and may even cause folding and collapse after surgery, affecting bone regeneration. If the modulus is too high, in one aspect, the modulus may not match that of the polymer material used for repair, and in another aspect, excessive stress is generated on a repair part, such that soft tissues are difficult to close and the metal is easy to expose, leading to infection.

Herein, the term “desired bending strength” refers to a strength enabling effective bending without breaking during the bone defect repair. The strength is generally 10-100 MPa, preferably 12-80 MPa, more preferably 13-50 MPa, and further preferably 15-20 MPa. The bending strength range can effectively support the composite film, maintaining a stable space.

Herein, the term “composite film” refers to an electroactive titanium scaffold-reinforced composite film, sometimes also referred to as an electrically responsive bone defect repair film, which is used for maintaining a space in a bone defect area to provide a space for osteoinducible growth for bone repair, and is particularly a film material suitable for alveolar bone augmentation to provide conditions for dental implant repair, which comprises a polymer material and a titanium scaffold coated therewith. The composite film generally has a thickness of 100-500 μm, preferably 120-400 μm, and more preferably 150-300 μm. The shape of the composite film is not particularly limited, and any shape may be designed according to clinical use. In an exemplary embodiment, the composite film is of a strip shape, and fixing sites are arranged corresponding to four corners of the strip shape or a vicinity thereof to retain fixing areas. The composite film comprises a titanium scaffold and a film material coating the titanium scaffold, which will be described in detail below.

### Titanium Scaffold

The titanium scaffold of the present application is used to prepare a composite film for use in bone augmentation, and has a comprehensive mechanical support structure designed according to the fixing sites of the composite film in use.

In the present application, the titanium scaffold generally comprises a main frame extending along a length direction and a secondary frame extending along a width direction. There are generally two secondary frames located at both ends of the main frame, respectively. The secondary frame is composed of a branched structure. The number of branched structures in each secondary frame is not limited, but at least a first branched structure and a second branched structure are included. If there are other branched structures, they are arranged between the first branched structure and the second branched structure. An included angle between the first branched structure and the second branched structure is not particularly limited, but is designed to ensure that an end of the first branched structure and an end of the second branched structure correspond to the fixing sites of the composite film or a periphery thereof or a vicinity thereof, respectively. For this reason, the preferred included angle is generally 20-40 degrees, preferably 22-38 degrees, and more preferably 24-26 degrees.

Preferably, the titanium scaffold of the present disclosure has an overall width of 8-18 mm and a length of 18-28 mm. More preferably, the titanium scaffold of the present disclosure has an overall width of 9-15 mm and a length of 19-25 mm.

In the present disclosure, the main frame and the secondary frame are composed of a titanium sheet or an elongated titanium strip, respectively, and the titanium sheet composing the main frame and the titanium sheet composing the branched structure have the same width, which is preferably 0.25-3 mm, and more preferably 0.35-1.5 mm.

The titanium scaffold of the present disclosure may be a flat structure or may be a customized or pre-bent structure.

In an exemplary embodiment, optionally, the titanium scaffold may be subjected to surface treatment or surface modification, for example, dopamine surface modification, surface roughening of the titanium scaffold, or the like. Further preferably, the dopamine surface modification can form a dopamine film on the surface of the titanium scaffold using dopamine by methods such as chemical oxidative polymerization, enzymatic oxidative polymerization, electrochemical polymerization, photopolymerization, or the like, so as to improve the biocompatibility of the titanium scaffold and promote bone formation. More importantly, the dopamine surface modification strengthens the bonding interaction or adhesive strength between the titanium scaffold and the polymer material layer, thereby preventing delamination in the film structure even under bending conditions when the film structure is used for bone augmentation. In a specific embodiment, the titanium scaffold is added to a 0.01-0.1 mol/L aqueous solution of dopamine, and the mixture is stirred for 6-12 h at 40-80° C., then ultrasonically vibrated for 1-15 min, washed by centrifugation 3-5 times, and then ultrasonicated for 1-10 min under a power condition of 180 W to obtain the dopamine-treated titanium scaffold.

In an exemplary embodiment, preferably, the surface roughening treatment may be performed using a sand blast-acid etching method. For example, the titanium scaffold is firstly subjected to sand blasting using SiO₂ particles under a pressure of 0.4 mPa, and then subjected to acid etching for 30 min using a mixed solution of 10% H₂SO₄ and 10% HCl at a constant temperature of 60° C.

In an exemplary embodiment, the titanium scaffold of the present application is of a dumbbell shape or substantially of a dumbbell shape, which is particularly suitable for preparing a rectangular composite film, in which case the titanium scaffold is preferably an integrally formed structure comprising a main frame extending along a length direction and two secondary frames extending along a width direction.

The main frame is an elongated strip-shaped structure, and the two secondary frames are located at both ends of the main frame, respectively, thereby forming a dumbbell shape or substantially a dumbbell shape. The structure has an up-down symmetrical structure and a left-right symmetrical structure. Each secondary frame is composed of two branched structures. An included angle formed by each two branched structures is 25 degrees. The length of the main frame is approximately 2 times the width of the secondary frame (i.e., a distance between the ends of the two branched structures).

When a repair film prepared based on the dumbbell-shaped titanium scaffold is used, fixing sites are arranged at positions corresponding to the first branched structure, the second branched structure, the third branched structure, and the fourth branched structure. A fixing hole may be arranged at the fixing site. For example, a through hole through which a fixing member passes may be arranged at the fixing site, and examples of the fixing member include, but are not limited to, a fixing bolt, and the like. The repair film based on the dumbbell-shaped titanium scaffold is particularly suitable for repair after the loss of a single anterior tooth. During use, the repair film can be bent along any direction, particularly along any symmetry axis of the titanium scaffold at both ends of the composite film.

In other exemplary embodiments, the titanium scaffold of the present application is of a pozidriv shape, which is particularly suitable for preparing a rectangular composite film, and the titanium scaffold is preferably an integrally formed structure comprising a main frame extending along a length direction and two secondary frames extending along a width direction.

The main frame is a titanium sheet with an elongated strip-shaped structure, and the two secondary frames are located at both ends of the main frame, respectively. Each secondary frame is composed of three titanium sheets, which form a first branched structure, a second branched structure, and a third branched structure, separately, wherein an included angle between the first branched structure and the second branched structure is 25 degrees, and the third branched structure is jointed with the main frame and forms an extension end of the main frame. The length of the extension end is equal to or substantially equal to that of the first or second branched structure.

In addition, a transverse frame is further arranged in the middle of the main frame along a direction perpendicular to the main frame. The length of the transverse frame is substantially equal to that of the main frame. The length of the main frame is substantially 2 times the width of the secondary frame (i.e., a distance between ends of the first branched structure and the second branched structure).

When a repair film for alveolar bone vertical augmentation prepared based on the pozidriv-shaped titanium scaffold is used, fixing sites are arranged at positions corresponding to the first branched structure, the second branched structure, and two branched structures on the opposite end that are symmetrical to the two branched structures. The repair film based on the pozidriv-shaped titanium scaffold is particularly suitable for repair after the loss of a single posterior tooth. During use, the repair film can be bent along any direction, particularly along any symmetry axis of the titanium scaffold at both ends.

In other exemplary embodiments, the titanium scaffold of the present application is of a glider shape, which is particularly suitable for preparing a rectangular composite film, and the titanium scaffold is preferably an integrally formed structure comprising a main frame extending along a length direction and two secondary frames extending along a width direction.

The main frame is a titanium sheet with an elongated strip-shaped structure, and the two secondary frames are located at both ends of the main frame, respectively. Each secondary frame is composed of two titanium sheets, which form a first branched structure and a second branched structure, wherein an included angle between the first branched structure and the second branched structure is 25 degrees.

A first transverse frame and a second transverse frame are further arranged in the middle of the main frame along a direction perpendicular to the main frame. The length of the first transverse frame is equal to that of the second transverse frame, and preferably, both are substantially equal to the length of the main frame. The length of the main frame is substantially 2 times the width of the secondary frame (i.e., a distance between ends of the first branched structure and the second branched structure).

When a repair film for alveolar bone vertical augmentation prepared based on the glider-shaped titanium scaffold is used, fixing sites are arranged at positions corresponding to four branched structures of the secondary frame. The repair film based on the glider-shaped titanium scaffold is particularly suitable for repair after the loss of multiple adjacent anterior/posterior teeth. During use, the repair film can be bent along any direction, particularly along any symmetry axis of the titanium scaffold at both ends.

### Film Material

The film material used in the present disclosure is a polymer material layer, wherein the polymer material includes PVDF and a derivative thereof, collagen, or chitosan, preferably PVDF and a derivative thereof. Examples of the polymer material layer include, but are not limited to, polyester, polyvinylidene fluoride PVDF, poly(vinylidene fluoride-trifluoroethylene) P(VDF-TrFE), polymethyl methacrylate PMMA, and polydimethylsiloxane. The polymer material layers on both sides of the titanium scaffold may be made of the same composition or different compositions. In certain embodiments, the polymer material layer may be dense, thereby preventing passage of bacteria or migration of connective tissue cells and epithelial cells therethrough. In other embodiments, the polymer material layer contains micropores, allowing oxygen or blood to pass through, while preventing the passage of bacteria or the migration of connective tissue cells and epithelial cells therethrough. Preferably, the film material forms a tight bond with the titanium scaffold of the present disclosure.

### Preparation Method

In a second aspect of the present application, provided is a preparation method for the electroactive titanium scaffold-reinforced composite film, which at least comprises:
(1) compositing the titanium scaffold inside a polymer material layer to form a film structure, and arranging sites or regions for fixing the film structure at positions corresponding to ends of bifurcations or vicinities thereof;
(2) increasing a temperature to 105-145° C., preferably 110-130°° C., and more preferably 120-130° C., at a rate of 2.5-4° C./min, keeping the temperature for 30-80 min, preferably 40-70 min, and more preferably 60 min, and then performing cooling, preferably natural cooling, to room temperature; and
(3) performing polarization treatment using a polarization method, with polarization treatment parameters comprising a polarization field intensity of 0.1-10 kV/mm and a polarization time of 10-60 min, such that the electroactive titanium scaffold-reinforced composite film can be obtained.

In step (1), the titanium scaffold can be cut by a known method, for example, by using a cutting device such as a laser micro-cutting machine. The thickness of the cut titanium substrate is generally 20-500 μm, such as 20-400 μm, preferably 20-200 μm. When a titanium substrate with a relatively high thickness is used, the substrate is preferably first subjected to thinning treatment, such as etching treatment. Generally, the surface of the titanium scaffold is roughened by the etching treatment, enhancing the adhesion to a polymer material layer, and thus the etching treatment is preferred.

In step (2), annealing treatment is performed, and the annealing assists in electrode polarizing, such that the obtained composite film material is uniformly and stably charged. The increase in the temperature of the surface of the composite film material can generate a pyroelectric effect, and the electrode polarizing can enable internal charges of the material to be polarized and deflected in a certain direction. The reason may be that after heating and cooling, the crystal generates surface charges in a certain direction due to temperature changes, and the polarization dipole moment can change with the direction of an applied electric field.

In step (3), high-voltage electric field polarizing enables the surface of the composite film to have a biomimetic electric potential, and a biomimetic electrical microenvironment is constructed in a damaged area. The polarization conditions include a polarization field intensity of 0.1-10 kV/mm, preferably 1-5 kV/mm, e.g., 2 V/mm, 3 V/mm, or 4 V/mm; and a polarization time of 5-60 min, preferably 10-50 min, and more preferably 15-40 min, e.g., 20 min, 25 min, 30 min, 35 min, etc.

In a specific embodiment, oil stains and dust are first removed from the surface of the titanium sheet substrate to keep the surface of the titanium sheet substrate smooth and clean, and the titanium sheet substrate is placed on a sample platform to be cut. Then, the dumbbell-shaped, pozidriv-shaped, or glider-shaped three-dimensional model file described above is designed. A moving path during cutting is set according to the three-dimensional model file, wherein the moving path forms the dumbbell shape, the pozidriv shape, or the glider shape described above, such that a manipulator cuts the titanium sheet substrate along the edge of the dumbbell shape, the pozidriv shape, or the glider shape. The process parameters for laser cutting are not particularly limited, and the parameters such as cutting speed, laser power, gas pressure, defocusing amount, working distance, cutting gas, etc. can be adjusted as required by those skilled in the art.

The process of forming the film structure is preferably achieved by the following steps: weighing out a ferroelectric polymer, adding the ferroelectric polymer to an organic solvent DMF, and stirring the mixture for 3-6 h until complete dissolution to obtain a polymer solution, wherein the concentration of the resulting solution is 1-5 g/ml, and the ferroelectric polymer is polyvinylidene fluoride or poly(vinylidene fluoride-trifluoroethylene); removing bubbles from the polymer solution in vacuum, pouring the polymer solution onto a quartz plate for drying, and obtaining a polymer film with a thickness of 10-500 μm after the organic solvent is completely volatilized; and placing the titanium scaffold or the dopamine-treated titanium scaffold between two polymer films, dissolving surface-layer polymers with, for example, DMF to bond the upper and lower films, and performing hot-pressing treatment until the two films are fully bonded to obtain the composite film material.

It should be noted that the film structure is fixed at the position corresponding to the end of the bifurcation or a vicinity thereof, thereby forming fixing sites at four corners of the film structure or a vicinity thereof, providing sufficient three-dimensional space for the regeneration of new bone under the condition of satisfying stress, and promoting osteogenesis. The relationship between exemplary fixing sites and the ends of the scaffold is as follows: both ends of the glider-shaped titanium scaffold are supported more, which facilitates the stress being transmitted from a stress point to the fixing bolts at both ends, and thus the overall stiffness of the glider-shaped titanium scaffold is higher. The dumbbell-shaped structure is similar to the pozidriv-shaped structure. Although a transverse frame is added to the pozidriv-shaped structure, fixing bolts are generally not added to both sides of the transverse frame, making the stress not transmitted. Therefore, the transverse frame does not provide significant support, and even the stiffness is lower due to deformation.

In the present disclosure, the electroactive titanium-reinforced composite film is preferably constructed by adopting a step-by-step casting method, and biomimetic charging of the titanium-reinforced composite film is realized by regulating and controlling conditions of the annealing treatment and polarization treatment. More preferably, the polarization treatment parameters are as follows: a polarization field intensity of 1 kV/mm, and a polarization time of 30 min, such that the electroactive titanium scaffold-reinforced composite film can be obtained.

In the present disclosure, the mechanical properties, such as tensile modulus, flexural strength, elastic modulus, etc., of the material can be determined by determination methods known in the art.

### Example 1

The preparation of an electroactive titanium scaffold-reinforced composite film is described in this example, specifically as follows:
(1) A pure titanium plate was fixed on a clamp to ensure flatness.
(2) The titanium plate was cut by using laser according to the designed three-dimensional model file.
(3) The titanium scaffold obtained in step (2) was ultrasonically cleaned in deionized water 3 times (5 min each time); the titanium scaffold was then put into absolute ethanol, ultrasonically cleaned 3 times (5 min each time), and dried to obtain a titanium scaffold, wherein the titanium scaffold is one of a dumbbell-shaped scaffold, a pozidriv-shaped scaffold, and a glider-shaped scaffold.

As shown in FIGS. 1 and 12, the dumbbell-shaped scaffold has an integrally formed structure, and comprises a main frame 110 extending along a length direction and two secondary frames 120 extending along a width direction. The main frame 110 has an elongated strip-shaped structure, and the two secondary frames 120 are located at both ends of the main frame 110, respectively, thereby forming the dumbbell shape. The structure has an up-down symmetrical structure and a left-right symmetrical structure. Each secondary frame 120 is composed of two branched structures. For example, the upper secondary frame 120 is composed of a first branched structure 121 and a second branched structure 122. The lower secondary frame 120 is composed of a third branched structure 123 and a fourth branched structure 124. An included angle formed by each two branched structures is 25 degrees. The length of the main frame 110 is approximately 2 times the width of the secondary frame (i.e., a distance between the ends of the two branched structures).

When a repair film for alveolar bone vertical augmentation prepared based on the dumbbell-shaped titanium scaffold is used, fixing sites are arranged at positions corresponding to the first branched structure 121, the second branched structure 122, the third branched structure 123, and the fourth branched structure 124. The repair film based on the dumbbell-shaped titanium scaffold is particularly suitable for repair after the loss of a single anterior tooth. During use, the repair film can be bent along any direction, particularly along any symmetry axis at both ends.

As shown in FIGS. 2 and 12, the pozidriv-shaped titanium scaffold has an integrally formed structure, and comprises a main frame 210 extending along a length direction and two secondary frames 220 extending along a width direction. The main frame 210 and the secondary frames 220 are composed of titanium sheets with the same width. The main frame 210 is a titanium sheet with an elongated strip-shaped structure, and the two secondary frames 220 are located at both ends of the main frame 210, respectively. Each secondary frame 220 is composed of three titanium sheets, which form a first branched structure 221, a second branched structure 222, and a third branched structure 223, wherein the included angle between the first branched structure 221 and the second branched structure 222 is 25 degrees, and the third branched structure is jointed with the main frame 210 and forms an extension end of the main frame 210. A transverse frame 211 is further arranged in the middle of the main frame 210 along a direction perpendicular to the main frame 210. The length of the transverse frame 211 is substantially equal to that of the main frame 210. The length of the main frame 210 is approximately 2 times the width of the secondary frame 220 (i.e., a distance between the ends of the first branched structure 221 and the second branched structure 222).

When a repair film for alveolar bone vertical augmentation prepared based on the pozidriv-shaped titanium scaffold is used, fixing sites are arranged at positions corresponding to the first branched structure 221, the second branched structure 222, and two branched structures on the opposite end that are symmetrical to the two branched structures. The repair film based on the pozidriv-shaped titanium scaffold is particularly suitable for repair after the loss of a single posterior tooth. During use, the repair film can be bent along any direction, particularly along any symmetry axis at both ends.

As shown in FIGS. 3 and 12, the glider-shaped titanium scaffold has an integrally formed structure, and comprises a main frame 310 extending along a length direction and two secondary frames 320 extending along a width direction. The main frame 310 and the secondary frames 320 are composed of titanium sheets with the same width. The main frame 310 is a titanium sheet with an elongated strip-shaped structure, and the two secondary frames 320 are located at both ends of the main frame 310, respectively. Each secondary frame 320 is composed of two titanium sheets, which form a first branched structure 321 and a second branched structure 322, wherein the included angle between the first branched structure 321 and the second branched structure 322 is 25 degrees. A first transverse frame 311 and a second transverse frame 312 are further arranged in the middle of the main frame 310 along a direction perpendicular to the main frame 310. The length of the first transverse frame 311 is equal to that of the second transverse frame 312, and both are substantially equal to the length of the main frame 310. The length of the main frame 310 is approximately 2 times the width of the secondary frame 320 (i.e., a distance between the ends of the first branched structure 321 and the second branched structure 322).

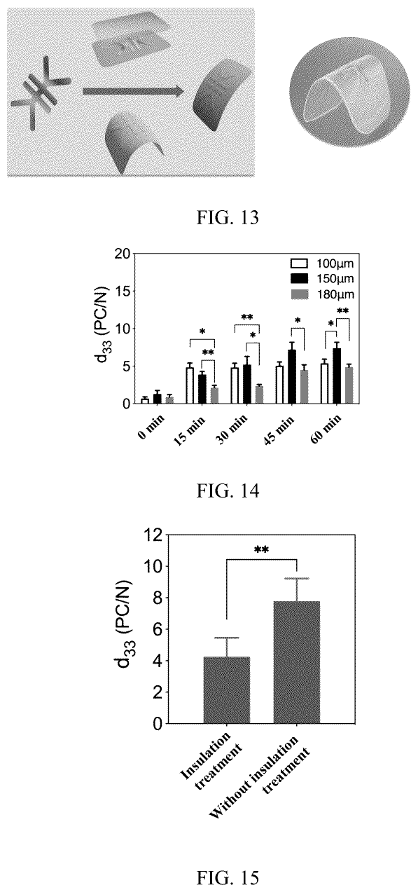

When a repair film for alveolar bone vertical augmentation prepared based on the glider-shaped titanium scaffold is used, fixing sites are arranged at positions corresponding to four branched structures of the secondary frame 320. The repair film based on the glider-shaped titanium scaffold is particularly suitable for repair after the loss of multiple adjacent anterior/posterior teeth. During use, the repair film can be bent along any direction, particularly along any symmetry axis at both ends.
(4) A certain amount of PVDF or a derivative thereof, such as P(VDF-Trfe), was poured into an organic solvent DMF (2 mL) for dissolving, and the resulting solution was stirred for 12 h and mixed uniformly; bubbles were removed in vacuum, and the resulting mixture was poured onto a quartz plate for drying; and a polymer film with a thickness of 50 μm was obtained after the organic solvent was completely volatilized.
(5) When the polymer film was not completely dried, the titanium scaffold obtained in step (2) was placed on the polymer film obtained in step (4), and then the mixed solution was poured to enable the titanium scaffold to be completely coated with the upper and lower films, such that the upper and lower films are fully bonded to obtain the titanium scaffold-reinforced composite film.
(6) The titanium scaffold-reinforced composite film obtained in step (5) was heated to 120° C. at a speed of 3.3° C./min, kept for 60 min, and naturally cooled to room temperature. The polarization treatment was performed by the annealing-assisted corona polarizing method, and the polarization treatment parameters are as follows: a polarization field intensity of 1 kV/mm, and a polarization time of 30 min, such that the electroactive titanium scaffold-reinforced composite film could be obtained (as shown in FIG. 13).
(7) Bone marrow mesenchymal stem cells were inoculated on the obtained electroactive titanium scaffold-reinforced composite film in a certain amount; the material prepared in Example 1 induced osteogenic differentiation of the stem cells; protein changes in an adhesion index (vinculin) and an osteogenic index (bone morphogenetic protein) were closely observed by using an immunofluorescence microscope, and it was observed that the bone marrow mesenchymal stem cells on the surface of the electroactive titanium scaffold-reinforced composite film showed significantly high expression of the vinculin and the bone morphogenetic protein; and then the material prepared in Example 1 was applied to critical mandibular defects of beagles, the bone regeneration effect was observed at three months by Micro CT quantitative analysis and H&E staining, and it was observed that there was a large amount of new bone regeneration in the defect areas covered by the electroactive titanium scaffold-reinforced composite film.

### Example 2

Another exemplary preparation of an electroactive titanium scaffold-reinforced composite film is described in this example. The differences from Example 1 are as follows: a stamping method was used in step (2) for processing, a hot-pressing method was used in step (5) to ensure full bonding of the two polymer material layers, and the polarization treatment parameters in step (6) were as follows: a polarization field intensity of 2 kV/mm, and a polarization time of 10 min. The scaffold was of a glider shape.

It was observed that the bone marrow mesenchymal stem cells on the surface of the electroactive titanium scaffold-reinforced composite film showed significantly high expression of the vinculin and the bone morphogenetic protein; and then the material prepared in Example 2 was applied to critical mandibular defects of beagles, the bone regeneration effect was observed at three months by Micro CT quantitative analysis and H&E staining, and it was observed that there was a large amount of new bone regeneration in the defect areas covered by the electroactive titanium scaffold-reinforced composite film.

### Example 3

Another exemplary preparation of an electroactive titanium scaffold-reinforced composite film is described in this example. The differences from Example 1 are as follows: a wire saw was used in step (2) for cutting, and the polarization treatment parameters in step (6) were as follows: a polarization field intensity of 5 kV/mm, and a polarization time of 60 min. The scaffold was of a glider shape.

It was observed that the bone marrow mesenchymal stem cells on the surface of the electroactive titanium scaffold-reinforced composite film showed significantly high expression of the vinculin and the bone morphogenetic protein; and then the material prepared in Example 3 was applied to critical mandibular defects of beagles, the bone regeneration effect was observed at three months by Micro CT quantitative analysis and H&E staining, and it was observed that there was a large amount of new bone regeneration in the defect areas covered by the electroactive titanium scaffold-reinforced composite film.

### Example 4

Another exemplary preparation of an electroactive titanium scaffold-reinforced composite film is described in this example. The differences from Example 1 are as follows: a metal 3D printing technology was used in step (2) for processing, and the scaffold was of a glider shape. The titanium surface was roughened, a hot-pressing method was used in step (5) to ensure full bonding of the two polymer material layers, and the polarization treatment parameters in step (6) were as follows: a polarization field intensity of 10 kV/mm, and a polarization time of 60 min.

### Example 5

Another exemplary preparation of an electroactive titanium scaffold-reinforced composite film is described in this example. The differences from Example 1 are as follows: a metal 3D printing technology was used in step (2) for processing, and the scaffold was of a glider shape. The titanium surface was treated with dopamine, a hot-pressing method was used in step (5) to ensure full bonding of the two polymer material layers, and the polarization treatment parameters in step (6) were as follows: a polarization field intensity of 10 kV/mm, and a polarization time of 60 min.

### Comparative Example 1

This comparative example is different from Example 1 in that annealing and polarization treatment were not performed in step (6).

Bone marrow mesenchymal stem cells were inoculated on the obtained titanium scaffold-reinforced composite film in a certain amount; the material prepared in Comparative Example 1 induced osteogenic differentiation of the stem cells; protein changes in an adhesion index (vinculin) and an osteogenic index (bone morphogenetic protein) were closely observed by using an immunofluorescence microscope, and it was observed that the bone marrow mesenchymal stem cells on the surface of the titanium scaffold-reinforced composite film could not better induce the spreading, adhesion, and osteogenic differentiation of the stem cells; and then the material prepared in Comparative Example 1 was applied to critical mandibular defects of beagles, the bone regeneration effect was observed at three months by Micro CT quantitative analysis and H&E staining, and it was observed that only a small amount of new bone was generated.

### Comparative Example 2

This comparative example is different from Example 1 in that the preparation process did not include the preparation of the polymer film in step (4).

Bone marrow mesenchymal stem cells were inoculated on the obtained titanium scaffold in a certain amount; the material prepared in Comparative Example 2 induced osteogenic differentiation of the stem cells; protein changes in an adhesion index (vinculin) and an osteogenic index (bone morphogenetic protein) were closely observed by using an immunofluorescence microscope, and it was observed that the bone marrow mesenchymal stem cells on the surface of the titanium scaffold could not better induce the spreading, adhesion, and osteogenic differentiation of the stem cells; and then the material prepared in Comparative Example 2 was applied to critical mandibular defects of beagles, the bone regeneration effect was observed at three months by Micro CT quantitative analysis and H&E staining, and it was observed that only a small amount of new bone was generated.

### Test Example 1

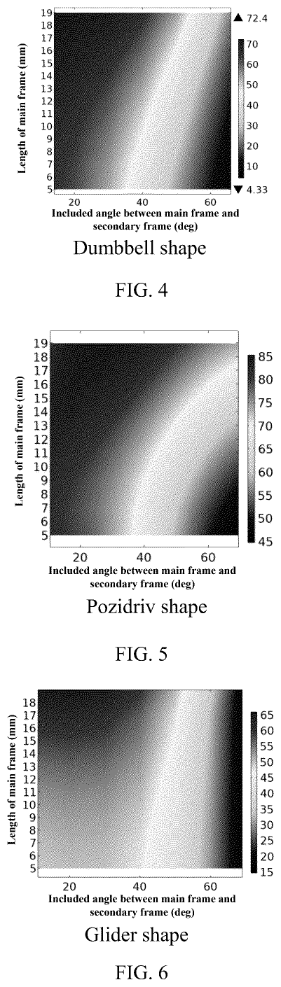

Three-dimensional finite element analysis was performed on the stress conditions of titanium scaffolds with different shapes in this test example. The results are shown in FIGS. 4-6. The research results show that for the dumbbell-shaped scaffold, the normal stiffness depends on the length of the main frame and the included angle between the secondary frame and the main frame. The smaller the included angle between the main frame and the secondary frame, the longer the length of the main frame. The change in the area above an angle of 30 degrees is relatively slow, and at an angle of 25 degrees, the main frame has higher stiffness. For the pozidriv-shaped scaffold, the transverse frame is added in the middle, and meanwhile, the middle support is provided at both ends, resulting in higher stiffness. Similar to the features of a single anterior tooth, a longer main frame and a smaller angle (25 degrees) enhance the normal stiffness of the structure. The glider-shaped scaffold can resist a relatively large lateral force and a vertical force, and when the transverse frame is close to both ends (30 degrees), the stiffness is higher.

Both ends of the glider-shaped titanium scaffold are supported more, which facilitates the stress being transmitted from a stress point to the fixing bolts at both ends, and thus the overall stiffness of the glider-shaped titanium scaffold is higher. The dumbbell-shaped structure is similar to the pozidriv-shaped structure. Although a transverse frame is added to the pozidriv-shaped structure, fixing bolts are generally not added to both sides of the transverse frame, making the stress not transmitted. Therefore, the transverse frame does not provide significant support, and even the stiffness is lower due to deformation. Simulations were performed on the titanium scaffolds in the above three forms. An identical normal load was loaded in the center of the titanium scaffolds with the same dimension, and the normal stiffness values of the scaffolds were 66.2 N/mm, 60.9 N/mm, and 83.9 N/mm, respectively. It can be seen that the glider-shaped titanium scaffold has the highest normal stiffness, and the optimal mechanical supporting effect is expected to be generated by the glider-shaped titanium scaffold.

### Test Example 2

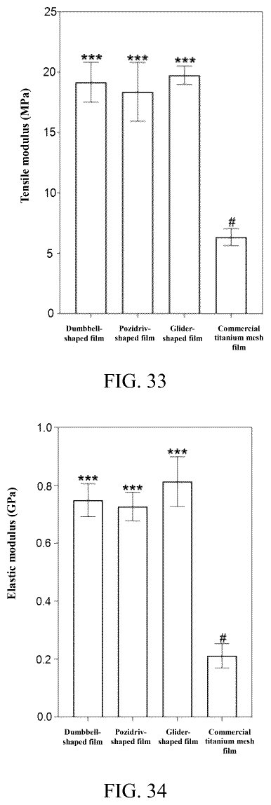

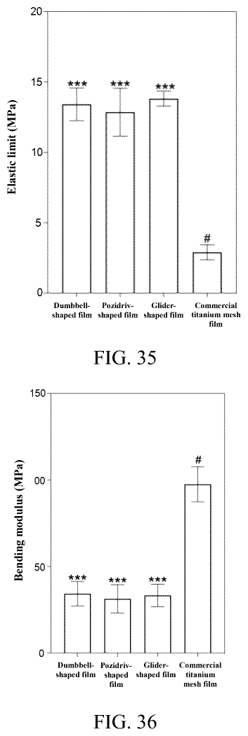

This test example shows the mechanical property characterization of the titanium scaffolds, and the results are shown in FIG. 7. The left panel in FIG. 7 shows the tensile strength results of the titanium scaffolds in different forms, and the right panel shows the bending strength results of the titanium scaffolds in different forms. The mechanical properties, including bending strength and tensile strength, of the pozidriv-shaped titanium scaffold and the glider-shaped titanium scaffold suitable for repairing the defect of the posterior tooth area, were significantly higher than those of the dumbbell-shaped titanium scaffold. The results of the property comparison data of the titanium scaffold composite films and the commercial titanium mesh composite film are shown in FIGS. 33-36. By comparing the dumbbell-shaped, pozidriv-shaped, and glider-shaped composite films with the commercial titanium mesh composite film, it was found that the glider-shaped composite film had higher elastic modulus, elongation at break, tensile strength, and elastic limit than those of the commercial titanium mesh composite film. The bending strength of the biomimetic electroactive titanium-reinforced composite films in different forms was lower than that of the commercial titanium mesh composite film. The composite film prepared by the traditional titanium mesh is not easy to bend due to the overlarge strength, making it difficult to shape according to the shape of the bone defect in clinical applications. Additionally, the bonding effect of the titanium mesh to polymers is poor, resulting in easy exposure of the titanium mesh.

### Test Example 3

As the mechanical strength of the titanium scaffold is far greater than that of a ferroelectric polymer P(VDF-TrFE) matrix, the mechanical strength of the biomimetic electroactive titanium-reinforced composite film mainly depends on the titanium scaffold. According to the research above, the mechanical properties of the glider-shaped titanium scaffold were determined to be optimal, such that the glider-shaped titanium scaffold was used in the design and construction of a biomimetic electroactive titanium-reinforced composite film material.

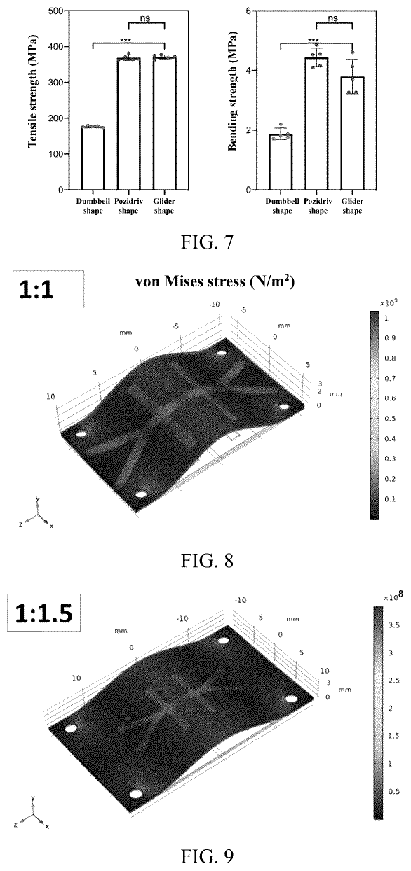

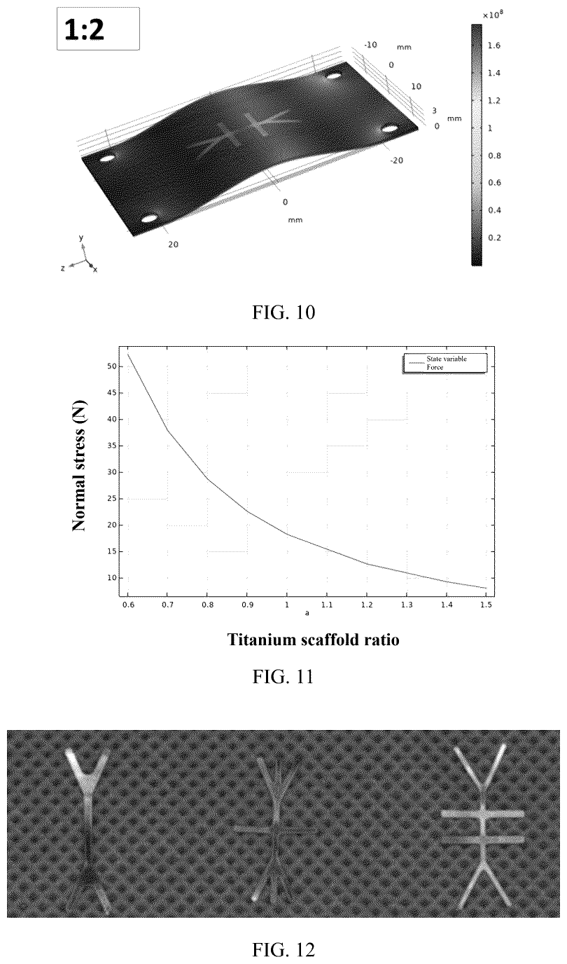

In order to investigate how the mechanical properties of the electroactive titanium-reinforced composite film were affected by the area ratio of the titanium scaffold in the ferroelectric polymer film, the area ratio of the titanium scaffold in the polymer film was designed and optimized. Through simulation and calculation, it was found that the smaller the area ratio of the titanium scaffold in the electroactive titanium-reinforced composite film, the poorer the mechanical support of the composite film. The area ratio was non-linearly and negatively correlated with the mechanical strength, with the optimal mechanical support of the composite film observed at a ratio of 1:1 (FIGS. 8-11).

### Test Example 4

In this test example, the thickness of the titanium scaffold in the composite film was optimized. Titanium scaffold composite films were prepared by setting titanium scaffolds with different thicknesses, and after polarization treatment, d₃₃ was measured. Comprehensively considering the chargeability (osteogenic inductivity) and plasticity (maintaining the shape of a bone defect area) of the material, a titanium foil with a thickness of 50 μm was selected for subsequent experiments. On this basis, the total thickness of the titanium-reinforced composite film was optimized and screened in the present disclosure. Titanium-reinforced composite films with thicknesses of 100 μm, 150 μm, and 180 μm were prepared, respectively, and the annealing times were set to be 0 min, 15 min, 30 min, 45 min, and 60 min. The piezoelectric constant of the titanium-reinforced composite film was detected, and it was found that the piezoelectric constant of the titanium-reinforced composite film with the film thickness of 150 μm was the highest under the condition that the annealing time was 60 min (FIG. 14), and the electrical level was consistent with the physiological magnitude range. Therefore, these parameter conditions were selected as the optimal parameters for subsequent research.

### Test Example 5

The interface performance of the titanium scaffold in the polymer matrix was optimized in this test example, so as to further improve the compatibility of the titanium scaffold and the polymer matrix and improve the electrical stability of the material. The surface of the titanium scaffold was subjected to insulation treatment by using an insulation treatment agent. Compared with the titanium scaffold without the insulation treatment, it was found that d₃₃ of the group without the insulation treatment was remarkably higher than that of the insulation treatment group (FIG. 15), and the piezoelectric constant d₃₃ was consistent with the biomimetic magnitude level.

### Test Example 6

The study on the physical and chemical performance of the electroactive titanium scaffold-reinforced composite film is described in this test example.

1. Mechanical properties of electroactive titanium scaffold-reinforced composite film

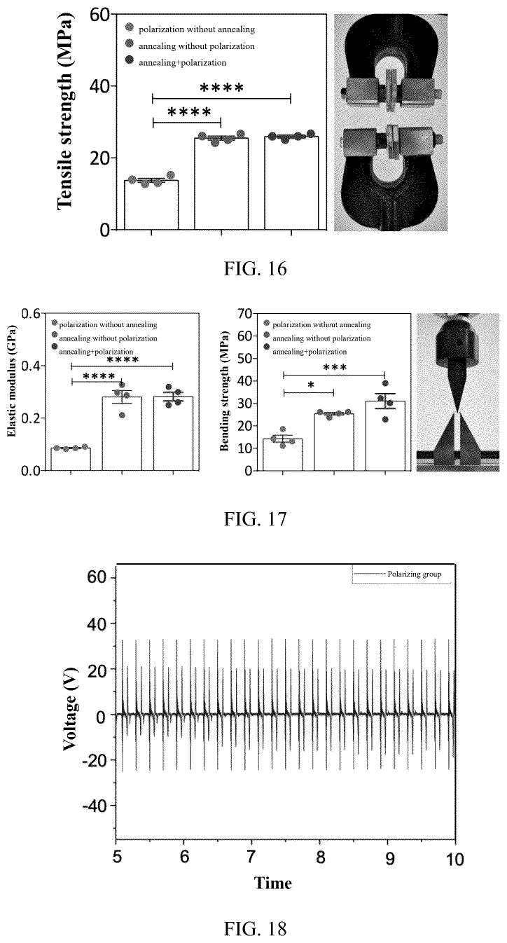

The titanium scaffold and the electroactive film material were compounded, such that not only the material was endowed with good plasticity, but the mechanical properties of the material could be effectively improved, providing a better mechanical supporting effect. The mechanical properties of electroactive titanium-reinforced composite films subjected to different treatment processes were systematically characterized in the present disclosure. The results show that the mechanical properties of the composite films, including tensile strength, elastic modulus, and bending strength, could be significantly improved by the annealing treatment and the corona polarization treatment combined with the annealing treatment (FIGS. 16-17).

### 2. Electrical Properties of Electroactive Titanium Scaffold-Reinforced Composite Film Material

### 2.1 Electromechanical Responsiveness of Electroactive Titanium Scaffold-Reinforced Composite Film Material

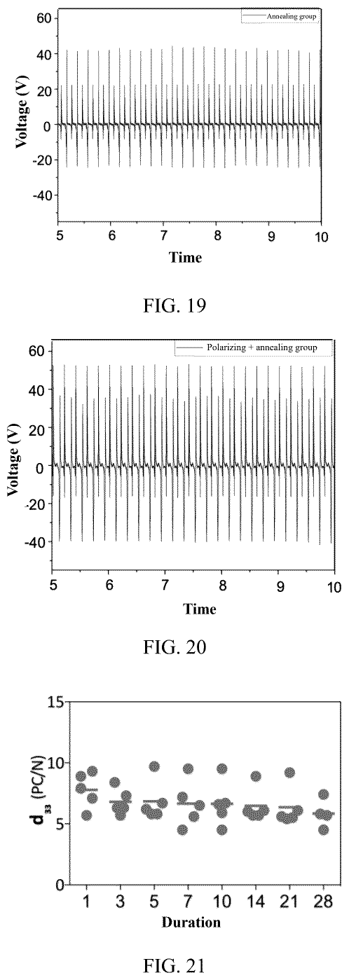

In order to further investigate the electrical responsiveness of the electroactive titanium-reinforced composite film, the electromechanical responsiveness of the electroactive titanium-reinforced composite film was evaluated in the present disclosure. A titanium-reinforced composite film was first fixed on polyacrylamide, and electrodes were prepared on the upper and lower surfaces of the titanium-reinforced composite film. Reciprocating bending motion was performed on the sample by using a loading motor, and an oscilloscope was used to characterize the voltage signal output of the material. It could be seen that the polarized titanium mesh-reinforced composite film exhibited stronger voltage output signals (FIGS. 18-20).

### 2.2 Electrical Stability of Electroactive Titanium Scaffold-Reinforced Composite Film Material

Considering that a defect needs to be repaired for a period of time when the electroactive titanium-reinforced composite film material is implanted into the defect, the evaluation of the electrical stability of the electroactive titanium-reinforced composite film is very important for the osteoinductive function of the electroactive titanium-reinforced composite film. In the present disclosure, a mode of incubation in a serum-free cell culture medium at 37° C. was adopted in vitro to simulate physiological conditions in vivo, and material samples were taken out at different time points to perform piezoelectric constant detection. The results show that the piezoelectric constant d₃₃ of the electroactive titanium-reinforced composite film after annealing and corona polarization treatment was 6-9 pC/N, which is consistent with the physiological piezoelectric constant level of bone tissue, and the piezoelectric constant d₃₃ of the electroactive titanium-reinforced composite film still kept good electrical stability after being incubated under the in vitro simulated condition for 28 days (FIG. 21).

### 3. In Vitro Biological Performance Evaluation of Electroactive Titanium Scaffold-Reinforced Composite Film

### 3.1 Promoting Adhesion of BMSCs and Cytoskeleton Rearrangement by Electroactive Titanium Scaffold-Reinforced Composite Film Material

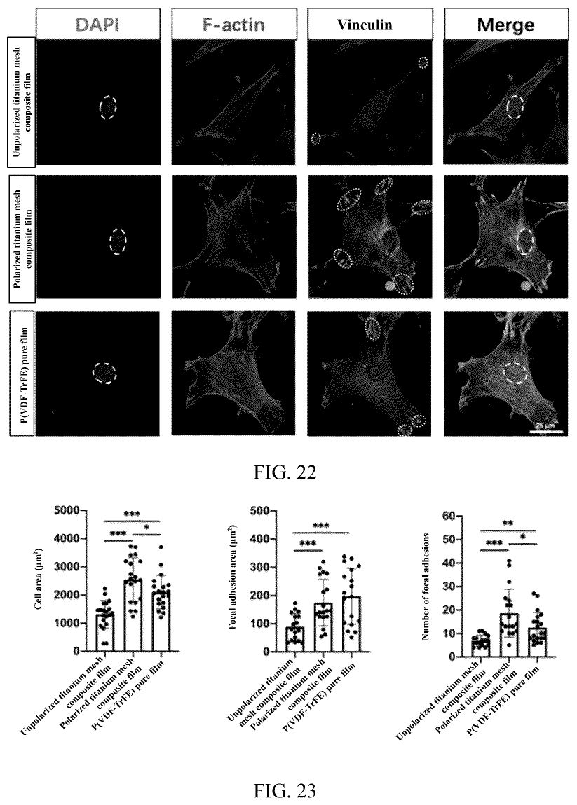

In order to evaluate the promotion effect of the electroactive titanium-reinforced nano-composite film material on the early adhesion of BMSCs, focal adhesions (Vinculin) and cytoskeleton (F-actin) were stained in the present disclosure. Firstly, bone marrow mesenchymal stem cells were inoculated on the surface of a material for 6 h, and then the cell spreading area and the cell adhesion state were observed. The cells were immobilized in 4% paraformaldehyde, and permeabilized with 0.3% Triton-X100. The non-specific binding sites of the cells were blocked with 3% BSA, and then focal adhesion antibodies were added for labeling specific antigens, DAPI was used for labeling cell nuclei, and FRITC-labeled phalloidine was used for labeling the actin cytoskeleton. The treated sample was observed under a confocal laser scanning microscopy. The results show that the formation of focal adhesions of BMSCs on the surfaces of the polarized titanium-reinforced composite film and the polarized P(VDF-TrFE) pure film was enhanced, the cells spread into a polygonal shape, and the spreading area was increased, which were superior to those of the unpolarized titanium-reinforced composite film group (FIGS. 22 and 23). The results indicate that the electroactive titanium-reinforced composite film material can significantly promote the adhesion of the bone marrow mesenchymal stem cells and cytoskeleton rearrangement, which is beneficial to the osteogenic functional differentiation of the bone marrow mesenchymal stem cells in the later period. The focal adhesion, as an important medium for contacting cells with materials, has important significance on the adhesion, proliferation, and functional differentiation of cells.

### 3.2 Inducing Osteogenic Differentiation of BMSCs by Electroactive Titanium Scaffold-Reinforced Composite Film Material

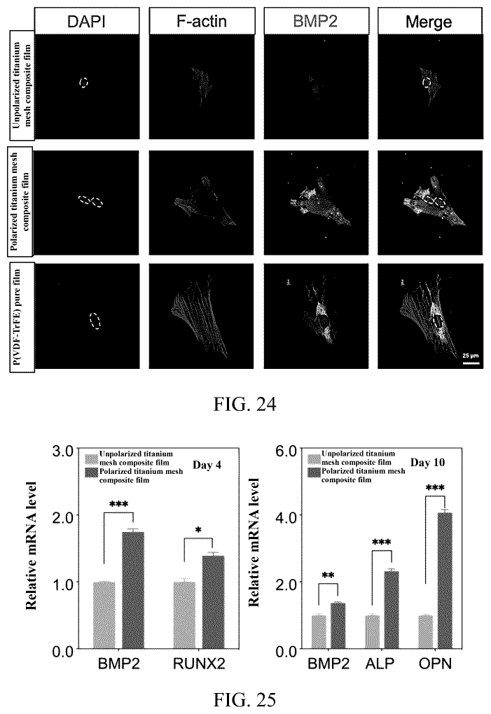

In order to explore the effect of the electroactive titanium scaffold-reinforced composite film on the osteogenic differentiation of bone marrow mesenchymal stem cells of rats, the present disclosure detected the protein levels of osteogenic differentiation-related markers by using an immunofluorescence technique. After the bone marrow mesenchymal stem cells of the rats were co-cultured with the electroactive titanium-reinforced composite film for 3 days, a cell osteogenic differentiation marker BMP2 was detected by immunofluorescence. The cells were immobilized in 4% paraformaldehyde, and permeabilized with 0.3% Triton-X100. The non-specific binding sites of the cells were blocked with 3% BSA, and then the BMP2 antibody was added for labeling specific antigens, DAPI was used for labeling cell nuclei, and FITC-labeled phalloidine was used for labeling the actin cytoskeleton. The treated sample was observed under a confocal laser scanning microscopy. The results show that both the polarized titanium-reinforced composite film and the polarized pure film could promote the high expression of BMP2 (FIG. 24), and the results indicate that the electroactive titanium-reinforced composite film can promote the osteogenic differentiation of the mesenchymal stem cells.

The present disclosure further detected the osteogenic differentiation markers at a gene level. After the bone marrow mesenchymal stem cells were co-cultured with the electroactive titanium-reinforced composite film for 4 days and 10 days, the expression levels of osteogenic genes (RUNX2, BMP2, ALP, and OPN) in the bone marrow mesenchymal stem cells of the rats were detected by real-time fluorescence quantitative PCR. The results show that the polarized titanium-reinforced composite film promoted the high expression of RUNX2 and BMP2 on day 4, and the expression levels of the osteogenic genes, ALP and OPN, were significantly up-regulated on day 10 (FIG. 25), indicating that the electroactive titanium-reinforced composite film exhibits excellent osteogenesis induction activity and can effectively induce the osteogenic differentiation of the mesenchymal stem cells in early, middle, and late stages.

### 4. Evaluation of Effect of Electroactive Titanium Scaffold-Reinforced Composite Film on Promoting Bone Defect Repair

### 4.1 Construction of Alveolar Bone Vertical Bone Augmentation Model in Beagles And Material Implantation

In the present disclosure, beagles were used as an experimental animal model to construct a vertical bone augmentation model after tooth extraction of alveolar bone. Ten healthy male beagles aged 12 months were taken, fasted for 12 h before operation, and anesthetized with a combination of Sumianxin and pentobarbital sodium. After the success of general anesthesia, conventional skin preparation, disinfection, and draping were done. Local anesthesia was performed on an operation area by using a 4% articaine-epinephrine injection; the gingiva was separated, and a sulcular incision and vertical incisions were made to incise the gingiva; a full-thickness flap was turned over; a power system was used to divide the premolar at the root furcation into mesial and distal parts; a periodontal membrane separator was used to detach the periodontal membrane; minimally invasive dental forceps were used to extract the teeth by occlusal traction; the alveolar fossa was curetted and rinsed with sterile normal saline; and an implant bed was prepared. The surgical animals were given free access to food after being awake. The liquid diet was given within 15 days, and clear water was fed after meals. Analgesic drugs (ibuprofen/tramadol, 50 mg/mL, 3 mg/kg Q12 h) were given for the first 3 days each week, an anti-inflammatory drug (meloxicam, 2 mg/20 kg) was given for the first 5 days, and antibiotics (spiramycin 750,000 IU/10 kg and metronidazole 125 mg/10 kg) were given for the first 10 days. 0.12% chlorhexidine was used for gargle to control plaque and avoid affecting wound healing. Three months after tooth extraction, a critical-sized alveolar bone defect model (FIG. 26) was prepared at the extraction sites on both sides of the mandible. The dimensions were 8 mm vertically, 11 mm mesiodistally, and 10 mm buccolingually. Bio-Oss bone powder was then filled into the defect, and the experimental film material was covered. A pure charged P(VDF-TrFE) film and a foreign commercial titanium-reinforced PTFE composite film were used as controls, and the wound was sutured tightly with 4-0 absorbable sutures. The surgical animals were given free access to food after being awake. The liquid diet was given within 15 days, and clear water was fed after meals. Analgesic drugs (ibuprofen/tramadol, 50 mg/mL, 3 mg/kg Q12 h) were given for the first 3 days each week, an anti-inflammatory drug (meloxicam, 2 mg/20 kg) was given for the first 5 days, and antibiotics (spiramycin 750,000 IU/10 kg and metronidazole 125 mg/10 kg) were given for the first 10 days. 0.12% chlorhexidine was used for gargle to control plaque and avoid affecting wound healing. The animals were sacrificed 4 and 12 weeks after implantation of the material by injection of pentobarbital sodium in a lethal dose, and the mandible specimens of the animals were fixed in a 10% neutral formalin solution for subsequent detection.

4.2 μCT analysis of Electroactive Titanium Scaffold-Reinforced Composite Film After In Vivo Implantation

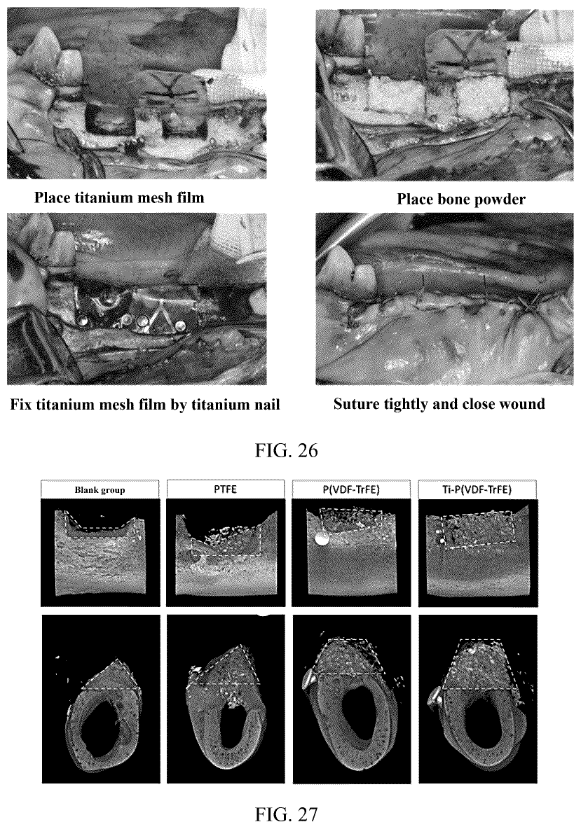

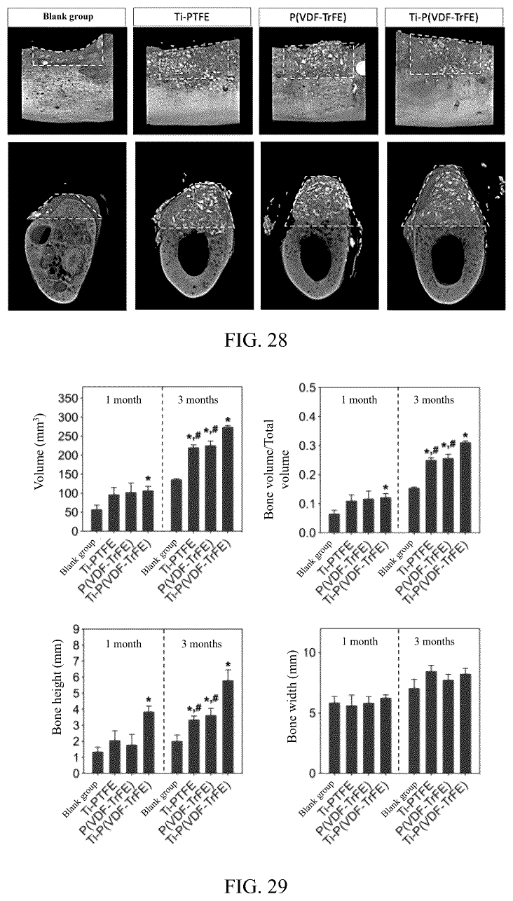

From the μCT results, it can be seen that the vertical bone augmentation and the new bone amount of the electroactive titanium scaffold-reinforced composite film group were significantly increased compared to the other three groups (FIGS. 27 and 28). The results of statistical analysis show that one month after operation, the vertical bone augmentation values of four groups, the blank group, the PTFE film group, the P(VDF-TrFE) film group, and the electroactive titanium scaffold-reinforced composite film group, were 1.36 mm, 2.07 mm, 1.80 mm, and 3.86 mm, respectively; the new bone amounts of the four groups were 57.7 mm³, 96.9 mm³, 102.8 mm³, and 107.4 mm³, respectively; and the vertical bone augmentation and the new bone amount of the electroactive titanium scaffold-reinforced composite film were both significantly improved compared to the other three groups. Three months after operation, the vertical bone augmentation values of the four groups were 2.01 mm, 3.35 mm, 3.64 mm, and 5.81 mm, respectively; the new bone amounts of the four groups were 136.3 mm³, 220.5 mm³, 226.1 mm³, and 274.2 mm³, respectively; the vertical bone augmentation and the new bone amounts of the three material groups were significantly improved compared to the blank group, and simultaneously, the vertical bone augmentation and the new bone amount of the electroactive titanium-reinforced composite film group were also significantly improved compared to the pure film group and the PTFE group. The bone augmentation in the electroactive titanium scaffold-reinforced composite film group increased by 72.6% compared to the bone augmentation before implantation, and by 24.32% compared to the bone augmentation of the PTFE product film group, showing a significantly improved vertical bone augmentation effect (FIG. 29).

It is well known that the vertical bone augmentation is a key technical problem in the clinical implantation repair of the oral cavity, and the present disclosure realizes a good bone augmentation effect of the electroactive titanium-reinforced composite film on a large animal model, which indicates that the electroactive titanium scaffold-reinforced composite film has a predictable bone augmentation effect and good clinical application prospects.

### 4.3 Histological Analysis of Vertical Bone Augmentation Promoted by Electroactive Titanium Scaffold-Reinforced Composite Film

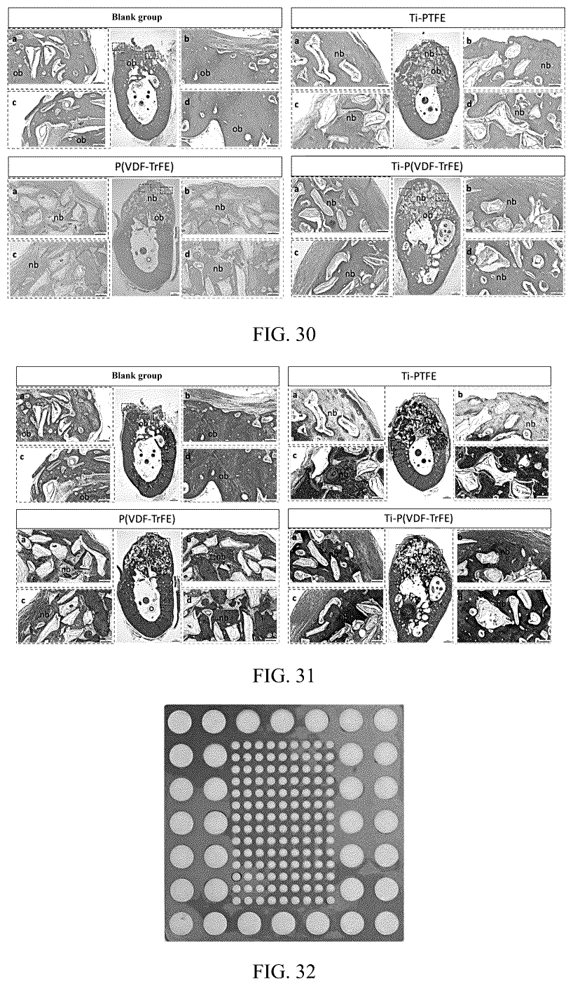

The histological staining results (FIGS. 30 and 31) show that 3 months after the implantation of the electroactive titanium scaffold-reinforced composite film, the new bone tissue was in a remodeling stage. The newly formed bone tissue had occupied the entire defect, the mineralization of the new bone was more active, resulting in the formation of more lamellar bone, which was thicker. The newly formed bone tissue in the entire defect area was less in the pure film group and the titanium PTFE group compared to the electroactive titanium-reinforced composite film. The bone defect of the blank group was not filled with the bone powder, which was found to be filled with a large amount of connective tissue, while less new bone tissue could be observed. In addition, the composite film of the present disclosure exhibits excellent anti-tissue adhesion properties. Particularly, animal experimental results show that after the composite film is conveniently removed from the micro-CT and histological specimens, the integrity of the repaired bone tissue is still maintained, and there is no residual tissue on the surface of the composite film. This indicates that the composite film of the present disclosure can effectively prevent tissue adhesion, thereby overcoming the shortcomings in the prior art that pure titanium meshes or existing expanded polymer repair film materials are prone to adhere to tissues.

Although the present disclosure has been described with reference to exemplary embodiments, it should be understood that the present disclosure is not limited to the disclosed exemplary embodiments. Various modifications and variations can be made to the exemplary embodiments of the specification of the present disclosure without departing from the scope or spirit of the present disclosure. The scope of the claims should be accorded the broadest interpretation so as to encompass all modifications and equivalent structures and functions.

[symbol] ridge preservation after tooth extraction.

1. An electroactive titanium scaffold-reinforced composite film, wherein, the composite film comprises: a titanium scaffold and a film material coating the titanium scaffold, wherein the titanium scaffold consists of a titanium-based material with a thickness of 20-500 μm and has a structure designed according to fixing sites;
the titanium scaffold comprises: a main frame and secondary frames attached to both sides of the main frame with a bifurcation structure, wherein the secondary frame comprises a first branched structure and a second branched structure at a certain angle, and ends of the bifurcation structure are located at the fixing sites or located at positions near the fixing sites.

2. The electroactive titanium scaffold-reinforced composite film according to claim 1, wherein the film material is a polymer material layer comprising a first layer and a second layer, and the titanium scaffold is coated with the first layer and the second layer, and an area ratio of the titanium scaffold in the composite film is 0.6-1.

3. The electroactive titanium scaffold-reinforced composite film according to claim 2, wherein the composite film has a quadrilateral or substantially quadrilateral profile, and the fixing site for fixing the composite film is arranged at each corner of the quadrilateral or a vicinity thereof; the main frame extends along a length direction, the secondary frame extends along a width direction, the main frame is an elongated strip-shaped structure, and the angle is 20-30 degrees, thereby forming the titanium scaffold into a dumbbell shape with a thin middle part and two wide ends.

4. The electroactive titanium scaffold-reinforced composite film according to claim 3, wherein the composite film is obtained by compositing the titanium scaffold inside the polymer material layer, annealing, and corona polarizing.

5. The electroactive titanium scaffold-reinforced composite film according to claim 2, wherein the first layer and the second layer each consist of identical or different compositions and are each independently selected from at least one of polyester, polyvinylidene fluoride PVDF, poly(vinylidene fluoride-trifluoroethylene) P(VDF-TrFE), polymethyl methacrylate PMMA, and polydimethylsiloxane.

6. The electroactive titanium scaffold-reinforced composite film according to claim 1, wherein the titanium scaffold further comprises a transverse frame located in the middle of the main frame and substantially perpendicular to the main frame.

7. The electroactive titanium scaffold-reinforced composite film according to claim 1, wherein the secondary frame further comprises a third branched structure located between the first branched structure and the second branched structure, and the third branched structure extends along a direction of the main frame, thereby forming the titanium scaffold into a pozidriv shape.

8. The electroactive titanium scaffold-reinforced composite film according to claim 1, wherein the titanium scaffold further comprises two transverse frames located at both ends of the main frame, respectively, and substantially perpendicular to the main frame, thereby forming the titanium scaffold into a glider shape.

9. The electroactive titanium scaffold-reinforced composite film according to claim 1, wherein the electroactive titanium scaffold-reinforced composite film has a thickness of 100-500 μm.

10. A preparation method for the electroactive titanium scaffold-reinforced composite film according to claim 9, comprising the following steps:
(1) compositing a titanium scaffold inside a polymer material layer to form a film structure, and arranging fixing sites at positions corresponding to ends of bifurcation structures of the titanium scaffold;
(2) increasing a temperature to 105-145° C. at a rate of 2.5-4° C./min, keeping the temperature for 30-80 min, and then performing cooling to room temperature; and
(3) performing polarization treatment using a polarization method, with polarization treatment parameters comprising a polarization field intensity of 0.1-10 kV/mm and a polarization time of 10-60 min, such that the electroactive titanium scaffold-reinforced composite film can be obtained.
